(19) United States
(12) Patent Application Publication
(10) Pub. No.: US 2011/0207779 A1
(43) Pub. Date: Aug. 25, 2011

(54) PROCESS FOR THE PREPARATION OF ESOMEPRAZOLE MAGNESIUM

(75) Inventors: Koilpillai Joseph Prabahar, Navi Mumbai (IN); Veerabhadra Swamy Hiremath, Navi Mumbai (IN); Milind Moreshwar Gharpure, Navi Mumbai (IN); Nagan Nirmalan Kandaswamy, Thane (IN); Avekumar Digambar Dabe, Nasik (IN); Kamal Mehta, Bhilwara (IN); Pankajkumar Shinde, Jalgaon (IN)

(73) Assignee: GLENMARK GENERICS LTD, Mumbai (IN)

(21) Appl. No.: 12/769,640

(22) Filed: Apr. 28, 2010

(30) Foreign Application Priority Data
Feb. 25, 2010 (IN) 506/MUM/2010

(51) Int. Cl. A61K 31/4439; C07D 401/12; A61P 1/04

(52) U.S. Cl. 514/338; 546/273.7

(57) ABSTRACT

The present invention relates to a process for the preparation of esomeprazole magnesium containing R-isomer greater than about 0.1% by wt. In particular, the present invention relates to a process for the preparation of esomeprazole magnesium dihydrate containing R-isomer greater than about 0.1% by wt.

### PRIORITY

This application claims priority to Indian Application IN 506/MUM/2010, filed Feb. 25, 2010, which is referenced herein in its entirety.

### BACKGROUND OF THE INVENTION

The present invention relates to a process for the preparation of esomeprazole magnesium containing R-isomer greater than about 0.1% by wt, specifically, esomeprazole magnesium dihydrate containing R-isomer greater than about 0.1% by wt and pharmaceutical compositions thereof.

Omeprazole is chemically known as 5-methoxy-2-[[(4-methoxy-3,5-dimethyl-2-pyridinyl)methyl]sulfinyl]-1H-benzimidazole. The S-enantiomer of omeprazole is chemically known as (S)-5-methoxy-2-[[(4-methoxy-3,5-dimethyl-2-pyridinyl)-methyl]sulfinyl]-1H-benzimidazole and is commonly referred to as “esomeprazole”. Esomeprazole is a well-known gastric proton-pump inhibitor used in the treatment of dyspepsia, peptic ulcer disease, gastroesophageal reflux disease and Zollinger-Ellison syndrome and has been commercially available from AstraZeneca under the brand name LOSEC®/PRILOSEC®.

The magnesium salt of esomeprazole in the form of trihydrate is marketed under the brand name NEXIUM® and is represented by Formula I.

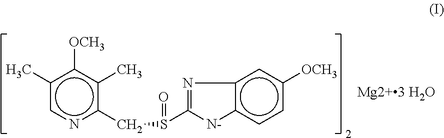

U.S. Pat. No. 5,714,504 describes alkaline salts of the (−) enantiomer of 5-methoxy-2-[[(4-methoxy-3,5-dimethyl-2-pyridinyl)methyl]sulfinyl]-1H-benzimidazoles (i.e., esomeprazole) including the magnesium salt.

U.S. Pat. No. 6,369,085 describes crystalline esomeprazole magnesium trihydrate, the crystalline dihydrate forms A, and B and processes for their preparation.

U.S. Pat. No. 6,875,872 (the '872 patent) describes magnesium salt of the (−) enantiomer of 5-methoxy-2-[[(4-methoxy-3,5-dimethyl-2-pyridinyl)methyl]sulfinyl]-1H-benzimidazole (esomeprazole magnesium) with an optical purity greater than about 99.8% enantiomeric excess (R-isomer is less than 0.1% by wt). The '872 patent discloses the preparation of esomeprazole magnesium containing high optical purity from a non aqueous medium by reaction of a crude esomeprazole with a methanolic magnesium methoxide followed by separation of the inorganic by-products and crystallization of the resultant esomeprazole magnesium from a mixture of methanol and acetone. This process entails use of excess volumes of crystallization solvent, such as acetone resulting in a process that is expensive and difficult to operate on an industrial scale.

It would be desirable to provide a process for the preparation of esomeprazole magnesium with pharmaceutically acceptable optical purity, which is simple and cost effective; in a convenient, cost efficient manner and a commercial scale.

### SUMMARY OF THE INVENTION

The present invention relates to a process for the preparation of esomeprazole magnesium containing R-isomer greater than about 0.1% by wt. In particular, the present invention relates to a process for the preparation of esomeprazole magnesium dihydrate containing R-isomer greater than about 0.1% by wt in a simple and commercially scalable method, which include the use of omeprazole magnesium as initiator to achieve the required quality of the product consistently.

The present invention provides a process for the preparation of esomeprazole magnesium containing R-isomer greater than about 0.1% by wt; comprising;
a) providing a solution of esomeprazole magnesium in an organic solvent;
b) adding omeprazole magnesium,
c) precipitating the resultant esomeprazole magnesium containing R-isomer greater than about 0.1% by wt.

The present invention provides a process for the preparation of esomeprazole magnesium containing R-isomer greater than about 0.1% by wt; comprising;
a) providing a solution of esomeprazole magnesium in an organic solvent;
b) adding omeprazole magnesium, wherein the omeprazole magnesium has an R-isomer content which is greater than about 0.3%.
c) precipitating the resultant esomeprazole magnesium containing R-isomer greater than about 0.1% by wt.
   wherein the organic solvent is selected from the group consisting of ethers selected from diethyl ether, tetrahydrofuran (THF), methyl tertiary butyl ether; alcohols selected from methanol, ethanol, isopropanol; water; and mixtures thereof.

The present invention provides, wherein the resultant esomeprazole magnesium has R-isomer content greater than about 0.2% by wt. Preferably, greater than about 0.5% by wt, more preferably greater than about 1% by wt.

The present invention provides esomeprazole magnesium having purity not less than 99.99% as measured by high performance liquid chromatography (HPLC).

The present invention provides esomeprazole magnesium having greater than about 0.1% by wt of R-isomer as measured by high performance liquid chromatography (HPLC).

The present invention provides a pharmaceutical composition comprising esomeprazole magnesium containing R-isomer greater than about 0.1% by wt, prepared by the process of the present invention, together with one or more pharmaceutically acceptable excipients.

### DETAILED DESCRIPTION OF THE INVENTION

The present invention relates to a process for the preparation of esomeprazole magnesium containing R-isomer greater than about 0.1% by wt. In particular, the present invention relates to a process for the preparation of esomeprazole magnesium dihydrate containing R-isomer greater than about 0.1% by wt in a simple and commercially scalable method. The present invention provides a process for the preparation of esomeprazole magnesium containing R-isomer greater than about 0.1% by wt, comprising;
a) providing a solution of esomeprazole magnesium in an organic solvent;
b) adding omeprazole magnesium,
c) precipitating the resultant esomeprazole magnesium containing R-isomer greater than about 0.1% by wt.

The starting esomeprazole magnesium in the directly described process may be prepared by any of known methods familiar to one of ordinary skill in the art. Illustratively, these methods are as those described in U.S. Pat. Nos. 5,714,504, 6,124,464; and 6,369,085, which are disclosed herein as references, in their entirety.

The starting esomeprazole magnesium, for example, may be prepared by the following method: dissolving a salt form of esomeprazole, preferably esomeprazole potassium salt in an organic solvent selected from methanol, methylene chloride, and mixtures thereof at a temperature at about 30° C. to about 35° C. Then the esomeprazole potassium salt solution may be treated with a magnesium source at temperature about 30° C. to about 35° C., wherein the magnesium source is magnesium sulfate heptahydrate, or magnesium chloride hexahydrate. After the completion of the reaction, the inorganic by-products formed during the reaction are filtered and the filtrate is concentrated to obtain the esomeprazole magnesium, which is the starting material of the present invention.

The starting esomeprazole magnesium may also be prepared by starting from esomeprazole free base, which is converted to esomeprazole magnesium by treatment with a magnesium source; or by the direct conversion of esomeprazole from other alkaline salt forms then to esomeprazole magnesium by known methods.

The organic solvent that can be used in a) of the process described above, for the preparation of solution of esomeprazole magnesium is selected from the group consisting of ethers selected from diethyl ether, tetrahydrofuran (THF), methyl tertiary butyl ether; C₁₋₄ alcohols selected from methanol, ethanol, isopropanol, t-butanol; water; and their mixtures thereof in various proportions without limitation. Preferably the organic solvent is mixture of methyl tertiary butyl ether, isopropanol and water. The ratio of solvents methyl tertiary butyl ether, isopropyl alcohol and water is from about 0.2:0.2:0.05 to about 5:5:1 preferably the ratio is 0.45:0.45:0.1.

The temperature for the preparation of solution in a) of the process can range from about 25° C. to about 65° C., preferably from about 25° C. to about 35° C. The time period for the preparation of solution can range from about 30 minutes to about 5 hours. Preferably, from about 30 minutes to about 1 hour.

The omeprazole magnesium which can be used in b) of the process described above, is any form of omeprazole magnesium, for example any hydrated or solvated omeprazole magnesium can be used. The omeprazole magnesium can be used from a range of about 0.3 to about 1% by wt of the starting esomeprazole.

The omeprazole magnesium can be added either along with addition of the starting esomeprazole magnesium in an organic solvent or it may be added after the starting esomeprazole magnesium solution is formed in an organic solvent. The time of addition of omeprazole magnesium is not a criteria which needs monitoring, either modes of addition arrives at the appropriate result.

The omeprazole magnesium can also be added to the starting esomeprazole free base in an organic solvent in presence of a magnesium source; or added to the starting esomeprazole alkaline salts in an organic solvent in presence of a magnesium source to preparation of the esomeprazole magnesium of the present invention.

The time period for the precipitation of esomeprazole magnesium can range from about 30 minutes to about 6 hours. Preferably from about 1 hour to about 5 hours.

The esomeprazole magnesium can be recovered by any conventional technique known in the art, for example filtration. Typically, if stirring is involved, the temperature during stifling can range from about 10° C. to about 35° C. Preferably at about 20° C. to about 30° C., more preferably at about 25° C. to about 30° C.

The resultant product may optionally be further dried. Suitably, drying can be carried out in a tray dryer, vacuum oven, air oven, fluidized bed drier, spin flash dryer, flash dryer and the like. The drying can be carried out at a temperature ranging from about 30° C. to about 90° C. Preferably, at a temperature of about 50° C. to about 60° C.

The drying can be carried out for any desired time until the required product purity is achieved, e.g., a time period ranging from about 1 hour to about 20 hours. Preferably about 10 hours.

The esomeprazole magnesium recovered using the process of the present invention is esomeprazole magnesium dihydrate having R-isomer content of greater than about 0.1% by HPLC.

The present invention provides an advantageous process for preparing esomeprazole magnesium. For instance, the process of the instant invention described herein, circumvents the use of large volumes of crystallization solvents for example, about 30 volumes w/v of methanol and acetone as described in the '872 patent. In contrast, the process herein described the use of about 5 volumes w/v of mixture of methyl tertiary butyl ether, isopropanol and water. Moreover, the present invention described herein, advantageously and consistently reproduces the esomeprazole magnesium with a pharmaceutically acceptable optical purity using omeprazole magnesium as seed crystals.

A process for preparation of esomeprazole magnesium containing R-isomer between the range of about 0.1 to 1% by wt, comprising:
a) providing a solution of starting esomeprazole salt in an organic solvent;
b) adding omeprazole magnesium in an amount between the range of about 0.3 to 1% by wt of the starting esomeprazole,
c) precipitating the resultant esomeprazole magnesium containing R-isomer between the range of about 0.1 to 1% by wt.

The process described above, wherein the solution of esomeprazole salt comprises esomeprazole free base, and magnesium source in the organic solvent.

Esomeprazole magnesium containing R-isomer between the range of about 0.1 to 1% by wt as measured by chiral HPLC, prepared by process previously described.

A pharmaceutical composition comprising esomeprazole magnesium containing R-isomer between the range of about 0.1 to 1% by wt, prepared by the process described above, together with one or more pharmaceutically acceptable excipients.

Advantageously, the compound of the present invention, prepared by the process herein described exists in a well defined and stable state, which allows easier characterization and facile handling and storage. Additionally, the compound, prepared by the process herein described, is easier to synthesize in a reproducible manner and thereby easier to handle in a full scale production.

In yet another embodiment, the present invention provides esomeprazole magnesium dihydrate having a purity not less than about 99.8% as measured by chiral high performance liquid chromatography (HPLC).

In a still further embodiment, the present invention provides esomeprazole magnesium dihydrate having purity of about 99.9%, as determined by chiral HPLC.

In another embodiment, the present invention provides esomeprazole magnesium dihydrate having greater than about 0.1% by wt of R-isomer, as determined by chiral HPLC; preferably greater than about 0.2%, more preferably greater than about 0.5%, still more preferably greater than about 1%.

In another embodiment, the present invention provides a pharmaceutical composition comprising the esomeprazole magnesium dihydrate containing R-isomer greater than about 0.1% by wt obtained by the process of present invention, as an active ingredient, in association with a pharmaceutically acceptable carrier, diluent or excipient and optionally other therapeutic ingredients. Useful in the manufacture of a medicament for use in the treatment of a gastric-acid related condition and a method of treating a gastric-acid related condition which method comprises administering to a subject suffering from said condition a therapeutically effective amount of the esomeprazole magnesium dihydrate according to the invention.

The pharmaceutical compositions of the invention include compositions suitable for oral or parental administration. The most preferred route is the oral route for example in the form of capsules, tablets, mini tablets, granules, pellets, multi-unit particulated system (MUPS). The compositions may be conveniently presented in unit dosage forms, and prepared by any methods known in the art of pharmacy.

The pharmaceutical composition of the present invention uses esomeprazole magnesium dihydrate containing R-isomer greater than 0.1% by wt and other required pharmaceutically acceptable excipients selected from amongst the diluents, water-soluble polymers, pH-buffering compounds, surface active agents, disintegrants, glidants and lubricants in the core. The water-soluble polymers are selected from the group consisting of polyvinyl pyrrolidone, hydroxypropyl methylcellulose, hydroxypropyl cellulose, hydroxyethyl cellulose, methylcellulose and polyethylene glycol. The diluents are selected from the group consisting of lactose, mannitol, dibasic calcium phosphate, microcrystalline cellulose, calcium sulphate, dextrates, dextrin, sucrose, sorbitol, calcium carbonate, magnesium carbonate, kaolin, maltodextrin, starches, pregelatinized starch and tricalcium phosphate.

The pH-buffering compounds are selected from the group consisting of compounds usually used in antacid formulations such as, for instance, magnesium oxide, hydroxide or carbonate, aluminium or calcium hydroxide, carbonate or silicate; composite aluminium/magnesium compounds such as, for instance Al₂O₃.6MgO.CO₂.12H₂O, (Mg₆Al₂(OH)₁₆CO₃.4H₂O), MgO.Al₂O₃.2SiO₂.nH₂O, aluminium hydroxide/sodium bicarbonate co-precipitate or similar compounds; or other pharmaceutically acceptable pH-buffering compounds such as, for instance the sodium, potassium, calcium, magnesium and aluminium salts of phosphoric, carbonic, citric or other suitable, weak, inorganic or organic acids; or suitable organic bases, including basic amino acids and salts thereof.

The disintegrants are selected from the group comprising of crospovidone, croscarmellose sodium, sodium starch glycolate, low substituted hydroxypropyl cellulose, starch and magnesium aluminium silicate. The glidants are selected from the group comprising of colloidal silicon dioxide and talc. The lubricants are selected from the group comprising of magnesium stearate, calcium stearate, hydrogenated vegetable oil, paraffin, polyethylene glycol, sodium benzoate, zinc stearate, stearic acid and talc.

Before applying enteric coating layer(s) onto the core material in the form of individual tablets, said tablets may optionally be covered with one or more seal coatings comprising pharmaceutical excipients optionally including alkaline compounds such as for instance pH-buffering compounds. This/these seal coating(s) separate(s) the core material from the outer layer(s), the outer layer(s) being enteric coating layer(s). The seal coating(s) can be applied to the core material by coating or layering procedures in suitable equipments such as coating pan, coating granulator or in fluidized bed apparatus using water and/or organic solvents for the coating process. As an alternative the seal coating(s) can be applied to the core material by using powder coating technique. The materials for seal coating(s) are pharmaceutically acceptable compounds such as, for instance, sugar, polyethylene glycol, polyvinylpyrrolidone, polyvinyl alcohol, polyvinyl acetate, hydroxypropyl cellulose, hydroxyethyl cellulose, methylcellulose, ethylcellulose, hydroxypropyl methylcellulose, carboxymethylcellulose sodium, carageenan, ammoniomethacrylate copolymer (Type A) and others, used alone or in mixtures. Additives such as channel forming agents, plasticizers, pH-buffering compounds, colorants, opacifiers, fillers, anti-tacking and anti-static agents, such as for instance magnesium stearate, titanium dioxide, talc and other additives may also be included into the seal coating(s). The plasticizers are selected from the group comprising of polyethylene glycol, triethyl citrate, diethyl phthalate, dibutyl phthalate, dibutyl sebacate, acetyl triethyl citrate, acetyl tributyl citrate, tributyl citrate, sorbitol and glycerol.

The anti-tacking agents are selected from the group comprising of talc, colloidal silicon dioxide and magnesium stearate and paraffin. The fillers are selected from the group comprising of lactose, mannitol, dibasic calcium phosphate, microcrystalline cellulose, calcium sulphate, dextrates, dextrin, sucrose, sorbitol, calcium carbonate, magnesium carbonate, kaolin, maltodextrin, starches, pregelatinized starch, tricalcium phosphate, talc and titanium dioxide. The coloring agents are selected from the group comprising of ferric oxides. The opacifier is titanium dioxide. When the optional seal coating(s) is applied to the core material it may constitute a variable thickness. The maximum thickness of the optional seal coating(s) is normally only limited by processing conditions. The seal coating(s) may serve as a diffusion barrier and may act as a pH-buffering zone. The pH-buffering properties of the seal coating(s) can be further strengthened by introducing into the layer(s) substances chosen from a group of compounds usually used in antacid formulations such as, for instance, magnesium oxide, hydroxide or carbonate, aluminium or calcium hydroxide, carbonate or silicate; composite aluminium/magnesium compounds such as, for instance Al₂O₃.6MgO.CO₂.12H₂O, (Mg₆Al₂(OH)₁₆CO₃.4H₂O), MgO.Al₂O₃.2SiO₂.nH₂O, aluminium hydroxide/sodium bicarbonate co precipitate or similar compounds; or other pharmaceutically acceptable pH-buffering compounds such as, for instance the sodium, potassium, calcium, magnesium and aluminium salts of phosphoric, carbonic, citric or other suitable, weak, inorganic or organic acids; or suitable organic bases, including basic amino acids and salts thereof. Talc or other compounds may be added to increase the thickness of the layer(s) and thereby strengthen the diffusion barrier.

One or more enteric coating layers are applied onto the core material or onto the core material covered with seal coating(s) by using a suitable coating technique. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s).

The enteric coating layers contain pharmaceutically acceptable plasticizers to obtain the desired mechanical properties, such as flexibility and hardness of the enteric coating layers. Such plasticizers are for instance, but not restricted to, triacetin, citric acid esters, phthalic acid esters, dibutyl sebacate, cetyl alcohol, polyethylene glycols, polysorbates or other plasticizers. Additives such as dispersants, colorants, pigments, polymers e.g. poly (ethylacrylate, methylmethacrylate), anti-tacking and anti-foaming agents may also be included into the enteric coating layer(s). Other compounds may be added to increase film thickness and to decrease diffusion of acidic gastric juices into the acid susceptible core.

To protect drug substance to obtain an acceptable acid resistance of the tablet dosage form according to the invention, the maximum thickness of the applied enteric coating layer(s) is normally only limited by processing conditions.

The process for the preparation of esomeprazole magnesium dihydrate containing R isomer greater than about 0.1% by wt of the present invention is simple, eco-friendly, robust, reproducible and easily scalable.

The examples which follow will further illustrate the preparation of the compound of the invention. These examples are not intended to limit the scope of the invention as defined hereinabove.

### EXPERIMENTAL

### Chiral High Performance Liquid Chromatography (Chiral HPLC)

The content of R-isomer in esomeprazole magnesium dihydrate was measured by chiral high performance liquid chromatography equipped with quaternary gradient pumps, variable wavelength UV detector attached with data recorder and integrator software under the following conditions:

| | |
|---|---|
| Column | Chiral-AGP, 100 × 4.0 mm, 5μ |
| Flow Rate | 0.6 ml/minute |
| Detection | UV 302 nm |
| Injection volume | 20 μL |
| Run time | 10 minutes |

Buffer preparation: mix 70 mL of 156.0 g/l solution of sodium dihydrogen phosphate R with 20 mL of a 179.1 g/l solution of disodium hydrogen phosphate R. Dilute with water to 1000 mL and then dilute 250 mL of this solution with water to 1000 ml.
Mobile phase: Acetonitrile: buffer solution pH 6.0 (65:435, v/v).

### High Performance Liquid Chromatography (HPLC):

The chemical purity of esomeprazole magnesium dihydrate was measured by high performance liquid chromatography equipped with quaternary gradient pumps, variable wavelength UV detector attached with data recorder and integrator software under the following conditions:

| | |
|---|---|
| Column | Intersil C₈-3, 125 × 4.6 mm, |
| | 5μ [Part No. 8HI50056] |
| Column temperature | 30° C. |
| Diluent | Mobile Phase |
| Flow Rate | 1.0 mL/minute |
| Detection | UV 280 nm |
| Injection Volume | 40 μL |
| Run time | 65 minutes |

Buffer preparation: dissolve 1.4 gm of disodium hydrogen phosphate anhydrous in 1000 ml of water and adjust pH to 7.7 with o-phosphoric acid.
Mobile Phase Buffer: Acetonitrile (76:24, v/v).

### EXAMPLES

### Example 1

### Preparation of Omeprazole Magnesium Dihydrate (for Seeding)

65 gms of omeprazole magnesium dissolved in 130 ml of methanol at temperature about 25° C. to about 30° C. to form a solution. 31.3 gms of magnesium sulphate heptahydrate was added to the resultant solution and stirred for 60 minutes at temperature about 25° C. to about 30° C. 260 ml of methylene dichloride (MDC) was added and stirred for about 60 minutes at temperature about 25° C. to about 30° C. Filtered the inorganic by products and washed with 65 ml of MDC. Filtrate was evaporated under vacuum at temperature about 40° C. to about 45° C. to obtain the oily product. 117 ml of methyl tertiary butyl ether, 117 ml of isopropanol, and 20 ml of water was added to the oily product and the contents were stirred for about 4 hours at temperature about 25° C. to about 30° C. The precipitated solid wad filtered and washed with 130 ml of isopropanol. The wet product was dried at temperature about 50° C. to about 55° C. under vacuum for about 12 hours to obtain 38 Gms of omeprazole magnesium dihydrate.

HPLC chemical purity: 99.98%

HPLC chiral purity: S-isomer: 49.86% and R-isomer: 50.14%

Moisture content: 5.62% w/w.

### Example 2

### Preparation of Esomeprazole Magnesium Dihydrate (Using 0.3% w/w of Omeprazole Magnesium as Seed Crystals)

25 gms of esomeprazole potassium was dissolved in 50 ml of methanol at temperature about 30° C. to about 35° C. 12 gms of magnesium sulphate heptahydrate was added to the resultant solution and stirred for about 60 minutes at temperature about 25° C. to about 30° C. 100 ml of MDC was added and stirred for 60 minutes at temperature about 25° C. to about 30° C. Filtered the inorganic by products and washed with 25 ml of MDC. Filtrate was evaporated under vacuum at temperature about 40° C. to about 45° C. to obtain the oily product. 45 ml of methyl tertiary butyl ether, 45 ml of isopropanol, 10 ml of water, and 0.075 gms of omeprazole magnesium dihydrate, obtained from example 1 was added to the oily product and the contents were stirred for 4 hours at temperature about 25° C. to about 30° C. The precipitated solid wad filtered and washed with 50 ml of isopropanol. The wet product was dried at temperature about 50° C. to about 55° C. under vacuum for about 12 hours to obtain 8.05 gms of esomeprazole magnesium dihydrate.

HPLC chemical purity: 99.99%

S-isomer: 99.76%

R-isomer: 0.24%

Moisture content: 5.35% w/w.

### Example 3

### Preparation of Esomeprazole Magnesium Dihydrate (Using 0.5% w/w of Omeprazole Magnesium as Seed Crystals)

25 gms of esomeprazole potassium was dissolved in 50 ml of methanol at temperature about 30° C. to about 35° C. 12 gms of magnesium sulphate heptahydrate was added to the resultant solution and stirred for about 60 minutes at temperature about 25° C. to about 30° C. 100 ml of MDC was added and stirred for about 60 minutes at temperature about 25° C. to about 30° C. Filtered the inorganic by products and washed with 25 ml of MDC. Filtrate was evaporated under vacuum at temperature about 40° C. to about 45° C. to obtain the oily product. 45 ml of methyl tertiary butyl ether, 45 ml of isopropanol, 10 ml of water, and 0.125 gms of omeprazole magnesium dihydrate, obtained from example 1 was added to the oily product and the contents were stirred for about 4 hours at about 25° C. to about 30° C. The precipitated solid wad filtered and washed with 50 ml of isopropanol. The wet product was dried at about 50° C. to about 55° C. under vacuum for about 12 hours to obtain 8.6 gms of esomeprazole magnesium dihydrate.

HPLC chemical purity: 99.99%

S-isomer: 99.60%

R-isomer: 0.39%

Moisture content: 6.42% w/w.

### Example 4

### Preparation of Esomeprazole Magnesium Dihydrate (Using 1.0% w/w of Omeprazole Magnesium as Seed Crystals)

25 gms of esomeprazole potassium was dissolved in 50 ml of methanol at about 30° C. to about 35° C. 12 gms of magnesium sulphate heptahydrate was added to the resultant solution and stirred for about 60 minutes at about 25° C. to about 30° C. 100 ml of MDC was added and stirred for about 60 minutes at about 25° C. to about 30° C. Filtered the inorganic by products and washed with 25 ml of MDC. Filtrate was evaporated under vacuum at about 40° C. to about 45° C. to obtain the oily product. 45 ml of methyl tertiary butyl ether, 45 ml of isopropanol, 10 ml of water, and 0.25 gms of omeprazole magnesium dihydrate, obtained from example 1 was added to the oily product and the contents were stirred for about 4 hours at about 25° C. to about 30° C. The precipitated solid was filtered and washed with 50 ml of isopropanol. The wet product was dried at about 50° C. to about 55° C. under vacuum for about 12 hours to obtain 7.2 gms of esomeprazole magnesium dihydrate.

HPLC chemical purity: 99.99%

S-isomer: 99.21%

R-isomer: 0.79%

Moisture content: 6.15% w/w.

### Example 5

### Composition for the Preparation of Esomeprazole Magnesium Dihydrate Capsules with Esomeprazole Magnesium Dihydrate Obtained from Example 2

TABLE 1
(i) Drug Loading on Base Pellets:

| S. No | Ingredients | Quantity |
|---|---|---|
| 1. | MCC Pellets(Celphere ® 305) | 3.0 gm |
| 2. | Esomeprazole magnesium dihydrate | 3.0 gm |
| 3. | PVP K-90 | 0.3 gm |
| 4. | Methanol* | q.s |

(ii) Seal Coating of the Drug Loaded Pellets (30%)

TABLE 2

| S. No | Ingredients | Quantity |
|---|---|---|
| 1. | Drug Loaded Pellets | 3.0 gm |
| 2. | Opadry II (White)* | 0.9 gm |
| 3. | Purified Water* | q.s. |

*Opadry II (white) used herein is either polyvinyl alcohol (PVA) based or Hydroxypropyle methyl based (HPMC)

(iii) Enteric Coating of the Seal Coated Pellets (30%)

TABLE 3

| S. No | Ingredients | Quantity |
|---|---|---|
| 1. | Seal coated Pellets | 3.0 gm |
| 2. | Eudragit L-30D 55^{#} | 0.9 gm |
| 3. | Talc^{@} | 0.45 gm |
| 4. | Triethyl Citrate (TEC)^{@} | 0.09 gm |

Esomeprazole magnesium dihydrate obtained from example 2 was loaded on the MCC Pellets using PVP K90, the drug loaded pellets were seal coated with Opadry II (containing either polyvinyl alcohol (PVA) or hydroxypropylene methyl based (HPMC)) by using Wurster process (Pam-Glatt GPCG 1.1). The seal coated pellets were enteric coated with Eudragit L 30 D 55 along with talc & TEC by using Wurster process (Pam-Glatt GPCG 1.1).

### Example 6

### Composition for the Preparation of Esomeprazole Magnesium Dihydrate Tablets with Esomeprazole Magnesium Dihydrate Obtained from Example 2

TABLE 4

| | | Qty/Mini-tablet | |
|---|---|---|---|
| S. No. | Ingredient | (mg) | |
| | Intra-granular (Pre-lubrication) | | |
| 1. | Esomeprazole magnesium dihydrate | 1.37 | |
| 2. | Mannitol (Pearlitol SD 200) | 2.85 | |
| 3. | Microcrystalline Cellulose (Avicel PH 200) | 3.33 | |
| 4. | Magnesium Oxide (Heavy) | 1.33 | |
| 5. | Hydrophobic colloidal silicon dioxide | 0.20 | |
| | (Aerosil R972) | | |
| 6. | Calcium CMC | 0.40 | |
| | Extra-granular (Lubrication) | | |
| 7. | Talc | 0.25 | |
| 8. | Calcium Stearate | 0.25 | |
| | Total | 10.0 | |
| | COATING - Seal Coating* | | |
| 9. | Opadry II White (PVA or HPMC Based | 1.50 | |
| | coating Material) | | |
| 10. | Purified Water | q.s. | |
| | COATING - Enteric Coating** | | |
| 11. | Eudragit L 30 D-55 | 1.72 | |
| 12. | Triethyl citrate | 0.01 | mg |
| 13. | Purified water | q.s. | |
| | Grand Total Weight | 13.22 | mg |

Esomeprazole magnesium dihydrate obtained from example 2 mixed with all intragranular and extra granular materials to compress into minitablets of 2.5 mm. The compressed minitablets then seal coated with Opadry II (containing either polyvinyl alcohol (PVA) or hydroxypropylene methyl cellulose (HPMC)) by using Wurster process (Pam-Glatt GPCG 1.1). The seal coated minitablets were then enteric coated with Eudragit L 30 D 55 along with talc & triethyl citrate (TEC) by using Wurster process (Pam-Glatt GPCG 1.1).

Experimental Results of Esomeprazole Magnesium Dihydrate

Input

| Sl. | Esomeprazole | Omeprazole | R |
|---|---|---|---|
| No | Potassium | Mg | Isomer |
| 1 | 25 g | 0.075 g | 0.24% |
| | | (0.3% w/w) | |
| 2 | 25 g | 0.1 g | 0.38% |
| | | (0.4% w/w) | |
| 3 | 25 g | 0.125 g | 0.39% |
| | | (0.5% w/w) | |
| 4 | 25 g | 0.1875 g | 0.72% |
| | | 0.75% w/w) | |
| 5 | 25 g | 0.25 g | 0.79% |
| | | (1.0% w/w) | |

Esomeprazole magnesium dihydrate containing R-isomer greater than about 0.1% by weight of the present invention remains within the range of R-isomer content even after formulated in above mentioned dosage forms when incubated at the recited temperature for the recited amount of time in a closed container.

1. A process for preparation of esomeprazole magnesium containing R-isomer between the range of about 0.1 to 1% by wt, comprising:
a) providing a solution of starting esomeprazole salt in an organic solvent;
b) adding omeprazole magnesium in an amount between the range of about 0.3 to 1% by wt of the starting esomeprazole,
c) precipitating the resultant esomeprazole magnesium containing R-isomer between the range of about 0.1 to 1% by wt.

2. The process of claim 1, wherein the solution of esomeprazole salt comprises esomeprazole free base, and magnesium source in the organic solvent.

3. Esomeprazole magnesium containing R-isomer between the range of about 0.1 to 1% by wt as measured by chiral HPLC, prepared by process according to claim 1.

4. A pharmaceutical composition comprising esomeprazole magnesium containing R-isomer between the range of about 0.1 to 1% by wt of claim 3, together with one or more pharmaceutically acceptable excipients.
